(19) United States
(12) Patent Application Publication
(10) Pub. No.: US 2010/0291219 A1
(43) Pub. Date: Nov. 18, 2010

(54) METHODS AND COMPOSITIONS RELATING TO PROGENITOR CELLS

(75) Inventors: Jeffrey M. Karp, Chestnut Hill, MA (US); Robert S. Langer, Newton, MA (US); Cecilia Graneli, Gothenburg (SE); Debanjan Sarkar, Watertown, MA (US)

Correspondence Address: WOLF GREENFIELD & SACKS, P.C., 600 ATLANTIC AVENUE, BOSTON, MA, 02210-2206, US

(73) Assignee: MASSACHUSETTS INSTITUTE OF TECHNOLOGY

(21) Appl. No.: 12/665,510

(22) PCT Filed: Jun. 20, 2008
(86) PCT No.: PCT/US08/07734

§ 371 Date: May 17, 2010

Related U.S. Application Data
(60) Provisional application No. 60/936,920, filed on Jun. 21, 2007.

(51) Int. Cl. A61K 9/14; C12N 5/071; A61K 35/12; C12M 1/02; C07K 14/745; A61P 43/00

(52) U.S. Cl. 424/489; 435/325; 424/93.7; 435/308.1; 530/382

(57) ABSTRACT

The invention provides compositions and methods for harvesting and enriching cells such as connective tissue progenitors cells using stirred bioreactors, and in some embodiments fibrin microcarriers.

### BACKGROUND OF THE INVENTION

There is a wide-spread interest in utilizing adult stem cells for tissue transplantation and regeneration. This is exemplified by the high occurrence of bone grafting procedures occurring each year in North America, making bone the most commonly transplanted tissue, second only to blood. Adult human mesenchymal stem cells derived from bone marrow are a promising source for tissue regeneration and repair. The use of adult stem cells avoids the ethical considerations surrounding the use of embryonic stem cells. In addition, adult stem cells allow for autologous transplant while embryonic stem cells do not. Strategies for utilizing autologous stem cells have traditionally relied on lengthy in vitro culturing steps to achieve sufficient cell expansion. Other challenges have included a lack of control over transplanted cells.

### SUMMARY OF THE INVENTION

The invention is premised in part on the surprising discovery that cells such as but not limited to connective tissue progenitor cells can be more efficiently isolated and enriched from a bodily sample by contacting the sample to a suspension of microcarriers in a stirred bioreactor. The stirred bioreactor employs microcarrier in solution, and these microcarriers therefore have a greater surface area available for contacting and binding cells than do unitary matrices of the prior art. Cells such as connective tissue progenitor cells bind to, and in some instances migrate into (i.e., impregnate), the microcarriers. The cells are intended, inter alia, for transplant into subjects together with the microcarriers, and optionally in the context of a polymer matrix into or onto which the cell-bound microcarriers are situated.

The invention is further premised in part on the discovery of a novel method for synthesizing (or generating or making or producing, as the terms are used interchangeably herein) fibrin microcarriers that are free of oil and free of organic solvents. The method for making these microcarriers does not rely on an oil-in-water emulsion step and therefore it does not require the use of organic solvents to remove any oils used to make such an emulsion. Fibrin microparticles of this nature are particularly suited for and compatible with use in humans. It has further been discovered in accordance with the invention that certain sizes of fibrin microcarriers are better suited for the attachment of cells including but not limited connective tissue progenitors.

The invention therefore generally provides composition and methods for isolating and enriching cells such as but not limited to connective tissue progenitor cells, as well as therapeutic uses for such cells. The invention further provides pharmaceutical compositions comprising enriched populations of cells including connective tissue progenitor cells, and optionally other therapeutic agents. The invention further provides devices and kits comprising the reaction vessels used for contacting samples and microcarriers (referred to herein as bioreactors), and optionally microcarriers, growth factor(s), medium, and/or instructions for use. The invention further provides non-emulsion methods for making oil-free and organic solvent free fibrin microcarriers, as well as the compositions made from such methods.

Thus, in one aspect, the invention provides a method for isolating cells such as connective tissue progenitor cells from a sample comprising contacting a sample with a plurality of microcarriers in solution under conditions and for a time sufficient for cells such as connective tissue progenitor cells in the sample to bind to the microcarriers, and harvesting from the solution microcarriers bound to cells such as connective tissue progenitor cells, wherein the sample is not cultured in vitro prior to or after contact with the microcarriers. In one embodiment, the majority of microcarriers within the plurality is porous. In another embodiment, the majority of microcarriers in solution is not in contact with more than one other microcarrier. It is to be understood that the methods provided herein are equally applicable to other cell types including but not limited to hematopoietic progenitor cells, and endothelial progenitor cells. For convenience and brevity, many embodiments are described in terms of connective progenitor cells. This is not however to be construed as limiting or narrowing the invention in any way.

In one embodiment, the sample is contacted with the plurality of microcarriers in a vessel. In one embodiment, the sample and the plurality of microcarriers in solution are continuously stirred during the contacting step. In one embodiment, the sample and solution are in a volume ratio of about 1:50.

In one embodiment, the microcarriers are harvested from the solution using density based separation. In another embodiment, the microcarriers are harvested from the solution using filtration.

In one embodiment, the sample is a freshly explanted sample. The sample may be a tissue or a fluid sample. The sample may be a blood sample, a bone marrow sample, an adipose tissue sample, a skin sample, a muscle sample, a cartilage sample, a blood sample, a lymph sample or a plasma sample. In one embodiment, the sample is a human sample.

In one embodiment, the plurality of microcarriers is homogeneous, while in another embodiment, the plurality of microcarriers is heterogeneous.

In one embodiment, one or more microcarriers in the plurality is comprised of fibrin, chitosan, hyaluronic acid, collagen, PLGA, alginate, agarose, poly(ethylene glycol) and dextran, methylcellulose, poly(acrylate), and/or poly(ester). In one embodiment, one or more microcarriers in the plurality is comprised of extracellular matrix, such as autologous extracellular matrix. In one embodiment, one or more microcarriers in the plurality is comprised of calcium phosphate, calcium carbonate, calcium sulphate, allograft bone, tricalcium phosphate, and/or hydroxyapatite.

In one embodiment, one or more microcarriers in the plurality comprises one or more agents. The one or more agents may be located on the surface of the one or more microcarriers and/or inside the one or more microcarriers.

In one embodiment, the one or more agents is a growth factor. The growth factor may be a fibroblast growth factor (FGF), epithelial growth factor, an insulin-like growth factor (IGF), a bone morphogenic protein (BMP), platelet derived growth factor (PDGF), a transforming growth factor, parathyroid hormone (PTH), a PTH related peptide, vascular endothelial growth factor (VEGF), a hedgehog protein, a hematopoietic colony-stimulating factor (CSF), osteoid-inducing factor (OIF), angiogenin, endothelin, hepatocyte growth factor, keratinocyte growth factor, ADMP-1, an interleukin, epithelial growth factor, dexamethasone, leptin, sortilin, transglutaminase, prostaglandin E, 1,25-dihydroxyvitamin D3, ascorbic acid, pro-collagen, glycerol phosphate, TAK-778, a statin, growth hormone, steel factor (SF), activin A (ACT), retinoic acid (RA), epidermal growth factor, a hematopoietic growth factor, erythropoietin, a tumor necrosis factor (TNF), an interferon, heparin binding growth factor (HBGF), nerve growth factor (NGF) or muscle morphogenic factor (MMP). The BMP may be BMP-2, BMP-3, BMP-4, BMP-6 or BMP-7. The growth factor may be transforming growth factor beta (TGF-β). In other embodiments, the one or more agents may be ascorbic acid or β-glycerophosphate. In still other embodiments, the one or more agents may be an anti-microbial agent, an anti-inflammatory agent, an immunosuppressive agent, or an inhibitor of bone resorption.

In one embodiment, one or more microcarriers in the plurality is pre-loaded with one or more agents.

In one embodiment, the one or more agents is a cell adhesion molecule. The cell adhesion molecule may be an antibody, an aptamer, a cell adhesive protein, a ceramic, a carbohydrate, a polysaccharide, or a lectin. The cell adhesive protein may be a positively charged protein such as poly-lysine, fibronectin, collagen, laminin, gelatin (denatured collagen), vitronectin, vascular cell adhesion molecule (V-CAM), intercellular adhesion molecule (I-CAM), tenascin, thrombospondin, osteonectin, osteopontin, bone sialoprotein, an RGD-containing peptide, cadherins, integrins, neural adhesion proteins, proline-rich proteins, poly-lysine, or hyaluronic acid.

In one embodiment, the contacting occurs for 60 minutes or less. In one embodiment, the contacting occurs at room temperature. In one embodiment, the contacting occurs in a solution volume of 1000 ml or less. In one embodiment, the contacting step occurs in the absence of exogenously added serum.

In one embodiment, the method enriches cells such as connective tissue progenitor cells 2-10 fold compared to the sample.

In one embodiment, the plurality of microcarriers has an average pore size of about less than or equal to 15 microns, less than or equal to 10 microns, less than or equal to 5 microns, or less than or equal to 1 micron.

In one embodiment, no hematopoietic cells are bound to the plurality of microcarriers. In another embodiment, hematopoietic cells represent less than 25%, less than 20%, less than 15%, less than 10%, or less than 5% of cells bound to the plurality of microcarriers.

In one embodiment, the plurality of microcarriers or a fraction thereof is comprised of a hydrogel. In one embodiment, the hydrogel is comprised of one or more of collagen, fibrin, and hyaluronic acid.

In one embodiment, the microcarriers harvested from the solution on average have at least one bound cell such as a connective tissue progenitor cell. In one embodiment, about 50% of the microcarriers harvested from the solution have at least one bound cell such as a connective tissue progenitor cell.

In one embodiment, a negative selection and/or a positive selection step is performed on the sample prior to contact with the plurality of microcarriers.

In some embodiments, the cells are isolated in less than 2 hours, or in less than 30 minutes.

In another aspect, the invention provides a method for transplanting cells such as connective tissue progenitors cells into a subject comprising isolating cells such as connective tissue progenitor cells from a sample according to any of the foregoing methods, and administering cells such as connective tissue progenitor cells into a subject in need thereof.

In one embodiment, the harvesting and administering steps are carried out intraoperatively. In one embodiment, the harvesting and administering steps are carried out within 2 hours.

In one embodiment, the sample is autologous to the subject. In another embodiment, the sample is allogeneic to the subject.

In important embodiments, the isolated or harvested cells such as connective tissue progenitor cells are administered to the subject by injection.

In another aspect, the invention provides a kit comprising a sterile vessel comprising an inlet/outlet port and a stirrer, and optionally instructions for isolating connective tissue progenitor cells from a sample according to any of the foregoing methods, wherein the microcarriers are contacted with the sample in the vessel.

The kit may further comprise a first container comprising a plurality of sterile microcarriers, a second container comprising one or more agents, and a third container comprising a sterile solution. The kit may further comprise a positive or negative selection device and/or reagents that facilitate positive or negative selection of a sample prior to contact with the microcarriers.

In one embodiment, the sterile vessel is battery operated. In one embodiment, the vessel is disposable. In other embodiments, the kit may comprise one or more selection devices or reagents such as antibodies, filters, columns, magnetic beads, and the like, for use in negative and/or positive selection of cells.

In still another aspect, the invention provides a pharmaceutical composition comprising cells such as connective tissue progenitor cells isolated according to of the foregoing methods and bound to microcarriers, and a pharmaceutically acceptable carrier.

In one embodiment, the pharmaceutically acceptable carrier is a polymer matrix. In another embodiment, the pharmaceutically acceptable carrier is a liquid carrier.

In one embodiment, the composition further comprises an anti-microbial agent, an anti-inflammatory agent, an immunosuppressive agent, and/or an inhibitor of bone resorption.

In one embodiment, the composition is formulated for injection, including for injection through a 14 gauge or smaller gauge needle.

In another aspect, the invention provides a non-emulsion method for making a fibrin microcarrier composition, comprising combining fibrinogen and thrombin to form a fibrin hydrogel, dehydrating the fibrin hydrogel, and grinding the dehydrated fibrin hydrogel to produce fibrin microcarriers.

In one embodiment, the fibrin microcarriers are free of organic solvent. In one embodiment, the fibrin microcarriers are free of oil.

In one embodiment, the method further comprises size separating the fibrin microcarriers. In one embodiment, the method further comprises isolating fibrin microcarriers having an average diameter of 85-180 μm. In one embodiment, the method further comprises exposing the fibrin microcarriers to a cross linking agent.

In one embodiment, the fibrin microcarriers are non-spherical.

In one embodiment, the fibrin hydrogel is lyophilized.

In another aspect, the invention provides a fibrin microcarrier made according to the foregoing methods.

In another aspect, the invention provides a composition comprising a plurality of fibrin microcarriers, at least 50% of which have an average diameter of 85-180 μm prior to hydration.

In one embodiment, at least 75% of the fibrin microcarriers have an average diameter of 85-180 μm. In one embodiment, at least 90% of the fibrin microcarriers have an average diameter of 85-180 μm.

In one embodiment, the fibrin microcarriers have a plurality of surface features that are 1-10 μm. In one embodiment, the fibrin microcarriers have a plurality of surface features that are 10-100 μm.

In one embodiment, the fibrin microcarriers are oil-free. In one embodiment, the fibrin microcarriers are free of organic solvent.

In one embodiment, the fibrin microcarriers comprise a cell adhesion molecule. In one embodiment, the cell adhesion molecule is a positively charged cell adhesion molecule. In one embodiment, the positively charged cell adhesion molecule is poly-lysine.

In one embodiment, the composition further comprises a pharmaceutically acceptable carrier. In one embodiment, the composition further comprises a mammalian cell.

In one embodiment, the fibrin microcarriers are cross-linked. In one embodiment, the fibrin microcarriers are non-spherical.

In still another aspect, the invention provides a method for producing cell-bound fibrin microcarriers comprising contacting a fibrin microcarrier made according to any of the foregoing methods with a cell for a time and under conditions sufficient for the cell to bind to the fibrin microcarrier.

In another aspect, the invention provides a method for producing cell-bound fibrin microcarriers comprising contacting a population of fibrin microcarriers made according to any of the foregoing methods with cells for a time and under conditions sufficient for the cells to bind to the fibrin microcarriers.

In one embodiment, contacting occurs in vitro. In one embodiment, contacting occurs by culturing the fibrin microcarrier with the cell.

In one embodiment, the fibrin microcarriers comprise a cell adhesion molecule. In one embodiment, the fibrin microcarriers comprise a positively charged cell adhesion molecule. In one embodiment, the fibrin microcarriers comprise poly-lysine. In one embodiment, the fibrin microcarriers are cross-linked.

In one embodiment, the cell is a connective tissue progenitor cell. In one embodiment, the cell is a hematopoietic progenitor cell. In one embodiment, the cell is an endothelial progenitor cell.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and/or the arrangement of components set forth in the following description or illustrated in the Figures. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE FIGURES

FIG. 1A is a schematic of one embodiment of the invention. The embodiment comprises harvesting a bone marrow aspirate from a subject (A), and placing the aspirate into a bioreactor that contains cell adhesive microcarriers (B). Connective tissue progenitor cells from the aspirate adhere to the microcarriers (C) and are isolated and combined with bone graft substitute for transplantation into, for example, a bone defect (D). Upon implantation, the microcarriers release growth factors which encourage differentiation of the bound progenitor cells into cells of various lineages and subsequent tissue regeneration.

FIG. 1B is a schematic of another embodiment of the invention. The embodiment comprises performing an initial separation (or selection) on a bone marrow aspirate obtained within the operating room. The separation is illustrated for example as passage through a filter to remove cells that not of interest (e.g., red blood cells) and to retain cells of interest. The population of larger cells is placed within a bioreactor containing pre-programmed cell adhesive microcarriers comprising one or more growth factors to control of cell fate post-transplantation (e.g., proliferation, differentiation, or matrix production). Connective tissue progenitors present in the bone marrow sample adhere to the microcarriers and can be easily injected or transplanted into a patient. Upon implantation, the microcarriers release growth factors which promote proliferation and induce differentiation of particular cells types leading to tissue generation.

FIG. 2 is a schematic of various agents that can be added to microcarriers. Ligands that enhance cell adhesion and/or growth factors may be coated onto the surface and/or incorporated within microcarriers (1). Cells such as connective tissue progenitor cells adhere to the external surface of microcarriers (2). After transplantation cells such as connective tissue progenitor cells on the microcarriers access the growth factors and differentiate accordingly.

FIG. 3 is a schematic demonstrating material properties of a microcarrier that can be modified to influence biological response upon transplant of the microcarrier into a subject.

FIG. 4 is a schematic overview of the fibrin microcarrier synthesis method.

FIG. 5A is a graph showing the percent of cells attached to the fibrin microcarriers of varying sizes as a function of time.

FIG. 5B is an image of a fibrin microcarrier in PBS.

FIG. 5C is a photograph of fibrin microcarriers after incubation with GFP labeled mesenchymal stem cells (hMSC) for 120 minutes. The Figure shows cells attached and stretched on the surface of a fibrin microcarrier.

FIGS. 6A-D is a series of images showing rapid stem cell attachment to pure fibrin microcarriers (i.e., fibrin microcarriers not comprising another agent such as poly-lysine) compared to poly-D-Lysine treated fibrin microcarriers observed within 30 min of combining the microcarriers and the cells (A). FIG. 6A shows pure microcarriers incubated with GFP-labeled human mesenchymal stem cells. FIG. 6B shows a phase contrast bright field image of the microcarriers and cells. FIG. 6C shows poly-D-Lysine coated fibrin microcarriers incubated with GFP hMSC. FIG. 6D shows a phase contrast bright field image. (Scale bar 150 μm.)

FIG. 7 shows data for the quantification of rapid mesenchymal stem cell attachment to pure fibrin microcarriers and poly-lysine treated fibrin microcarriers. Poly-lysine fibrin microcarriers showed over 3× more cell attachment within first 30 min.

It is to be understood that the Figures are not required for enablement of the invention.

### DETAILED DESCRIPTION OF THE INVENTION

The invention relates broadly to methods and compositions for isolating and enriching cells including but not limited to connective tissue progenitor cells, methods for using these cells and various other compositions in a transplant setting, and devices and kits for performing these methods. The methods provided herein use microcarriers to capture and retain (at least ex vivo) connective tissue progenitor cells, as well as other cell types including but not limited to hematopoietic and endothelial progenitors. The invention provides methods for making certain microcarriers as well as compositions of the microcarriers themselves. The invention contemplates the transplant of cells, microcarriers, or both (e.g., in a microcarrier-cell complex) to a subject in need thereof either as a stand alone therapy or in combination with scaffolds or grafts such as bone scaffolds or grafts, or other therapeutic agents. As an example, when administered to subjects these complexes are capable of regenerating tissue in vivo.

Connective tissue progenitor cells are multipotent cells that are able to differentiate into two or more of the osteogenic (bone), adipogenic (fat), myogenic (muscle), and chondrogenic (cartilage) lineages. An example of a connective tissue progenitor cell is a pericyte which is a smooth muscle-like cell that is comprised in the lining of blood vessels. The differentiative potential of these cells can be determined by culturing them in a variety of conditions. Culture conditions for the differentiation of connective tissue progenitor cells into a variety of tissues including bone, adipocyte, muscle and cartilage are described in U.S. Pat. No. 7,015,037 (Furcht et al.), the contents of which are incorporated herein by reference. Connective tissue progenitor cells are defined phenotypically by the expression of one or more mesenchymal progenitor cell markers such as STRO-1, CD13, CD44, CD73, CD90, CD105, CD166, CCR2b, prolyl 4-hydroxylase, and/or α-smooth muscle actin, and/or by the low or lack of expression of hematopoietic and endothelial cell markers such as CD11b, CD31, CD34, and CD45. One population of these cells has been defined phenotypically as CD34−, CD45− and CD44+.

The invention provides methods and compositions for isolating cells such as connective tissue progenitor cells from samples. As used herein, isolating cells means physically separating cells from their natural environment, including physical separation from one or more components of their natural environment. For example, a preparation of connective tissue progenitor cells isolated from a bone marrow sample may be one that has had some or all hematopoietic cells of the bone marrow sample removed. Preferably the ratio of connective tissue progenitor cells to hematopoietic cells is increased in the isolated preparation as compared to the starting sample. As another example, a preparation of connective tissue progenitor cells isolated from adipose tissue sample may be one that has had some or all adipose cells of the sample removed. As used herein, the terms isolating and harvesting are used interchangeably. Isolated populations of cells such as connective tissue progenitor cells however may still comprise a plurality of microcarriers particularly since the invention contemplates administration of the cells into a subject in the context of the microcarriers.

As used herein, enriching cells including for example connective tissue progenitor cells means increasing their frequency (for example, connective tissue progenitor cell per nucleated cell) compared to their frequency in the sample from which they were isolated. The methods provided herein are able to enrich cells such as connective tissue progenitor cells at least 2-fold, and more preferably more than 2-fold including at least 3-fold, 4-fold, 5-fold, 6-fold, 7-fold, 8-fold, 9-fold, 10-fold, 12-fold, 15-fold, 20-fold, or more. As an example, the frequency of connective tissue progenitor cells in the bone marrow is about 1 in 10,000 to 1 in 100,000 nucleated cells. The invention provides preparations from bone marrow having a frequency of connective tissue progenitor cells of at least 1 in 5,000 to 1 in 50,000 nucleated cells. Importantly, the invention also enhances the absolute number of cells such as for example in some embodiments connective tissue progenitor cells recovered from a sample, and thus the final preparation also contains a sufficient number of these cells for clinical applications.

The invention therefore provides methods for isolating (or harvesting) cells of interest including but not limited to connective tissue progenitor cells from samples. These methods require contacting the samples with microcarriers in solution. The contacting step involves interactions of the sample with the microcarriers in some embodiments when both are in flow. The contacting step may be performed in a vessel such as a bioreactor. At a minimum, the vessel has a port through which the sample and microcarriers enter and exit and a stirrer mechanism. The stirrer mechanism may be a paddle that runs about the length (or height) of the vessel, or is otherwise configured to ensure sufficient mixing of the vessel components during the contacting step. Other stirrer mechanisms include the use of a magnetic stirrer. Optionally, the rate of stirring (or mixing) in the vessel can be controlled. The vessel may also have a temperature control and a timer. The vessel may be battery operated so as to increase the ease of use, for example, in an operating room setting. Preferably the vessel contents are continuously stirred during the contacting step. It should be appreciated that conditions within the vessel may be modified in various ways to control aggregation of the microcarriers with or without cells. Conditions which may be modified include but are not limited to mixing speed, mixing time, volume of solution, concentration of microcarriers, and shape, number and configuration of the stirrer. The vessel may be referred to as a bioreactor herein. In some embodiments, the bioreactor may comprise a perfused bioreactor. In other embodiments, the bioreactor may comprise a NASA bioreactor, including a bioreactor that is placed and used in outer space or in other reduced gravity settings.

In other embodiments, contacting occurs in a solution that is not appreciably in flow including for example in a static culture.

The contacting step therefore may be varied, depending on the embodiment, with respect to time and temperature. When performed intraoperatively, it may be preferable to perform the contacting step at room temperature given that the time period between harvest of a sample and its subsequent re-introduction into a subject will be as short as possible. In some embodiments, the contacting step may be less than or equal to 2 hours, less than or equal to 1 hour, less than or equal to 50 minutes, less than or equal to 40 minutes, less than or equal to 30 minutes, less than or equal to 20 minutes, or less than or equal to 10 minutes. The total time from harvest of the sample to re-introduction into a subject will preferably be as short as possible, and may be equal to or less than 5 hours, equal to or less than 4 hours, equal to or less than 3 hours, equal to or less than 2 hours, or equal to or less than 1 hour. These times may also be the times between the harvest of a sample and the introduction of cells derived therefrom into the same or a different subject.

The invention contemplates performing the harvesting method intraoperatively. As used herein, the term intraoperatively means within the operating room (or thereabouts). The term does not refer to the transfer to the harvested sample to a distant physical location such as a laboratory separate from the operating ward. The ease of the harvesting method allows it to be performed while the subject is still in the operating room.

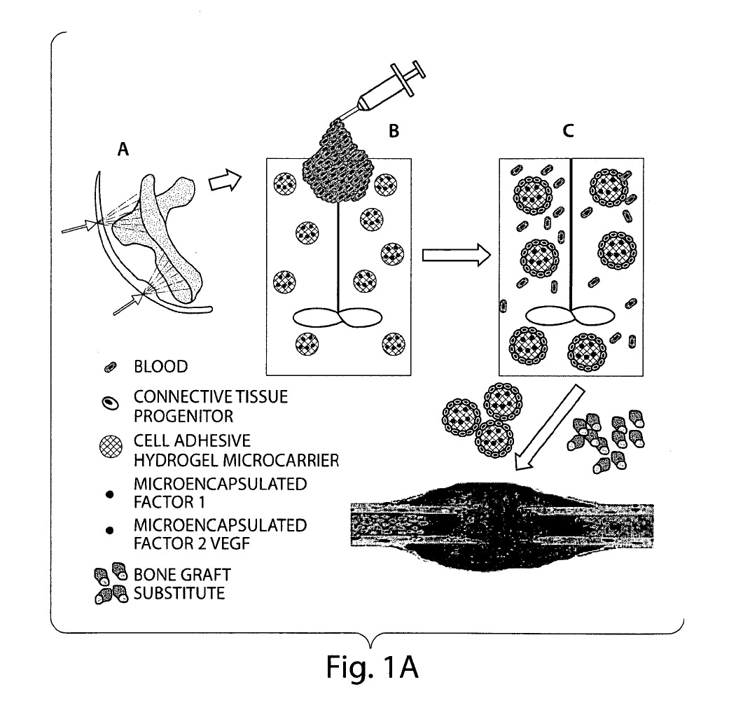

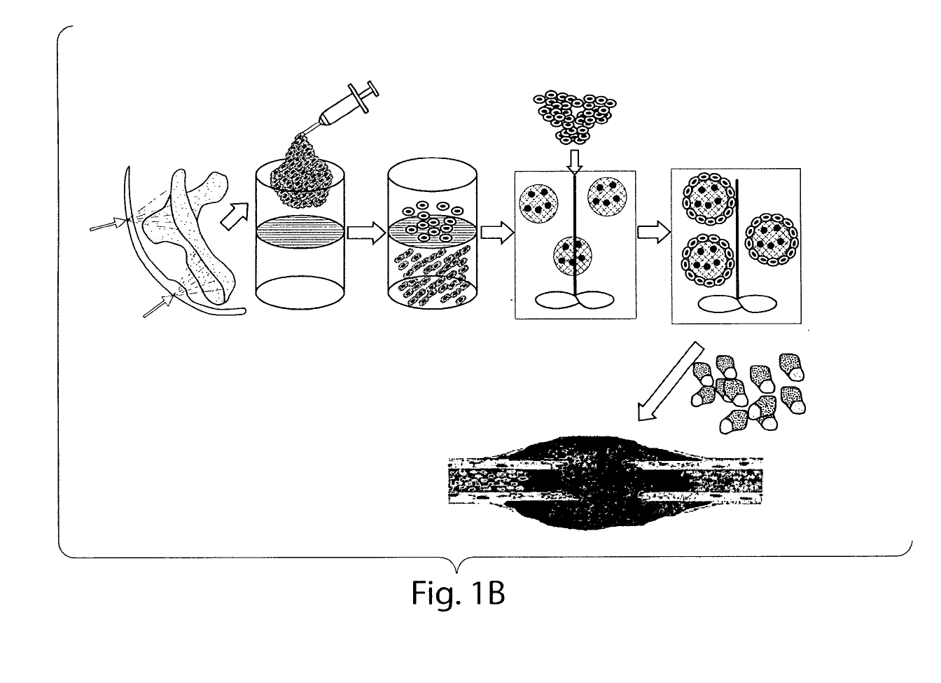

In most instances, the sample is harvested and directly introduced into a vessel that has already been loaded with microcarriers and solution. (FIG. 1A.) In the case of bone marrow, the sample is typically harvested with large gauge needle and so the invention contemplates that such needles can simply be evacuated (i.e., emptied) into the vessel, without an intervening transfer step. The volume of the vessel and the solution contained therein will depend on the volume of the harvested sample. The volume may therefore be expressed as a ratio of the solution volume to the sample volume. That ratio may be 1000:1, 500:1, 100:1, 50:1, 40:1, 30:1, 20:1 10:5:1, or 1:1. The volume may also be expressed as an absolute volume such as for example 1000 ml, 500 ml, 100 ml, or less.

In some embodiments, the sample is harvested from a subject and then introduced into the vessel without any intervening processing. Some samples may require some processing. For these samples, the processing can occur prior to entry into the vessel or within the vessel itself either prior to addition of the microcarriers or in the presence of the microcarriers. For example, adipose tissue samples may require collagenase treatment in order to release the progenitor cells. Such collagenase treatment can be performed on the tissue before it is added to the vessel, or within the vessel prior to microcarrier addition, or in the presence of microcarriers, with the proviso that such microcarriers are not susceptible to collagenase treatment (e.g., they do not comprise collagen).

The solution in which the microcarriers and sample are suspended and in which the contacting occurs may be any solution that is compatible with the cells with respect to isotonicity, pH, temperature, and the like, and which does not negatively impact the microcarriers. Preferably it does not comprise exogenously added serum. It may however comprise serum that is naturally present in the sample being added. The vessel should be contained so that the pH of the solution does not vary significantly.

The invention further contemplates negative selection steps prior to or after the contacting step. Negative selection refers to the separation and removal from a cell population of cells not of interest based on one or more features of these cells. These negative selection steps may for example be used to remove cells and/or components from the sample that may hinder contact and binding of the connective tissue progenitor cells with the microcarriers or that may impede successful transplant of these cells (e.g., the presence of certain hematopoietic cells in the final preparation to be administered may cause rejection problems). Negative selection procedures are known in the art, including the use of magnetic beads and particles for binding cells, “panning” where antibodies or other ligands for non-desired cells are immobilized on a plate to which cells are contacted in order to attach those non-desired cells, flow-through affinity columns loaded with similar antibodies or ligands, fluorescence activated cell sorting, and the like. As an example, if connective tissue progenitors are desired, then hematopoietic progenitors may be negatively selected based on their cell surface expression of CD34 and/or CD45.

The invention also further contemplates positive selection of cells prior to the contacting step in some embodiments. Positive selection refers to the separation and retention of cells of interest from other cells based on the features of the desired cells. Typically, positive selection is carried out based on the expression of one or more cell surface markers that may uniquely identify the cells of interest (or that may minimally enrich for these cells). Cell surface markers for connective tissue progenitors cells include those listed herein such as CD44, STRO-1, CD90, CD166, CD73, CD105, and CD29. Hematopoietic progenitors may be selected based on cell surface expression of CD34 and/or CD45. Endothelial progenitor cells may be selected based on cell surface expression of CD34, CD133 and VEGFR2.

Selection, whether positive or negative, may also occur based on other cell parameters including granularity, buoyant density, osmotic lysis susceptibility, and/or size. As an example, red blood cells are usually separable from other cells including connective tissue progenitor cells and hematopoietic progenitors based on size and buoyant density. (FIG. 1B.)

Kits and reagents (including magnetic beads, antibodies, ligands, and the like) for performing the foregoing methods are commercially available from sources such as Sigma, Becton Dickinson, Invitrogen, Chemicon, and Stem Cells Inc. These columns, filters, magnetic bead kits, antibody sets and the like are referred to herein as selection devices. Such devices can be included in the kits of the invention. Whether the device is a negative or a positive selection kit will depend on the nature of the reagents included therein as will be apparent to those of ordinary skill in the art.

As discussed herein, the microcarriers of the invention may be exposed to cell adhesion molecules, preferably prior to contact with cells. If the cell adhesion molecule is a cell non-specific cell such as a positively charged agent (e.g., poly-lysine), then in some instances it may be preferable to select for the cells of interest as much as possible prior to the contacting step. This is because the contacting step in this instance will not further select for particular cell types and the microcarriers would therefore be loaded with any cell type that is present. If on the other hand the cell adhesion molecule is cell specific such as certain antibodies or ligands, then in some instances positive selection may not be necessary prior to the contacting step since the contacting step itself will select for the cells of interest. However in these latter embodiments a negative selection may still be performed for example to remove cells that may interfere with the binding of the desired cells to the microcarrier or cells that bind non-specifically to the microcarriers (e.g., red blood cells).

Once the contacting step is complete, the microcarriers are harvested. It will be understood that harvesting the microcarriers will inherently also harvest any cells attached to thereto such as the connective tissue progenitor cells since these cells are bound to the microcarriers. It should also be understood that not all microcarriers may have cells such as progenitor cells bound to them. For example, depending on the contacting conditions, all or about 90%, about 80%, about 70%, about 60%, about 50%, about 40%, about 30%, about 20%, about 10% or less of the microcarriers harvested from the solution will have at least one bound cell such as a connective tissue progenitor cell. The microcarriers harvested from the solution may on average have at least 1, at least 2, at least 3, at least 4, at least 5, at least 10, at least 20, or more bound cells such as connective tissue progenitor cells. Regardless, the invention contemplates in some aspects transplanting all of these microcarriers into a subject.

The microcarriers (and thus cells) may be harvested by removing the solution from the vessel (via the same or a different port from that used to introduce these components) and physically separating the microcarriers from the solution. This physical separation can occur in any number of ways including density based separation (e.g., sedimentation by gravity, CCE, etc.), filtration (e.g., where the microcarriers are retained on a filter while the solution flows through), etc. The microcarriers are then collected and administered into the same or a different subject from which the initial sample was harvested. The microcarriers may be administered as is, or they may be introduced into a pharmaceutically acceptable carrier as described herein. In some instances, the microcarriers are combined with a polymer matrix that is then administered to a subject. The polymer matrix may be a hydrogel comprised of, for example, fibrin, collagen, alginate, hyaluronic acid and/or chitosan. Thus the microcarrier may be administered in solution or it may be administered within a solid or semi-solid gel. In important embodiments, the microcarriers are capable of being administered to subjects by injection. This increases the utility of the compositions of the invention as well as the ease of use.

The transplanting method of the invention comprises an administration step. As used herein, administering cells such as connective tissue progenitor cells into a subject implies that the microcarriers are also being administered to the subject. That is because the invention contemplates that cells such as connective tissue progenitor cells will continue to reside on, in, or in close proximity to the microcarriers in vivo, particularly if the microcarriers comprise agents that promote the growth and differentiation of the cells (such as for example some of the growth factors listed herein). Administration may be carried out by injection and/or implantation (i.e., placement of a preferably solid or semi-solid within a cavity, opening or wound). Injection or implantation may occur into or in the vicinity of bone, cartilage, muscle, skin or fat tissues, in some embodiments.

The harvested cells such as harvested connective tissue progenitor cells can be used in transplant therapies. Although not intending to be bound by any particular theory, the invention contemplates in one aspect that upon transplant into recipient subjects, connective tissue progenitor cells will produce an extracellular matrix akin to bone matrix. Thus, the microcarriers of the invention do not act as bone graft matrices or substitutes in some embodiments of the invention.

Subjects that would benefit from (and are therefore in need of) a connective tissue progenitor cell transplant include subjects having spinal fusions (e.g., to alleviate chronic back pain), temporomandibular joint reconstruction (e.g., to alleviate jaw pain), surgery to restore and/or reconstruct the contour and shape within craniofacial and maxillofacial bone, subjects having tibial as well as other bone fractures, subjects in need of orthopedic surgery and/or reconstruction, subjects having experienced bone trauma, elderly subjects, and subjects that have undergone radiation therapy, such as but not limited to cancer subjects being or having been treated in this manner.

Other subjects to be treated using the connective tissue progenitor cells are those having adult or congenital skin, bone, muscle, cartilage, or fat disorders. Bone disorders that weaken bone strength include but are not limited to bone cancer, osteoporosis or other low bone mass diagnoses, osteopetrosis, avascular necrosis, fibrous dysplasia, osteogenesis imperfecta, osteomyelitis, Paget's disease of the bone, and primary hyperparathyroidism. Other conditions which can be treated using the compositions of the invention include Crohn's disease, diabetes, Parkinson's disease, GVHD, and any degenerative disease affecting the bone, cartilage, muscle, skin or fat tissues. The therapeutic methods provided herein may be used on civilian and military populations and settings.

Subjects in need of hematopoietic progenitor cells include those that have undergone or are undergoing chemotherapy, radiation, or a bone marrow transplant. Subjects in need of endothelial progenitor cells include those that have undergone or are undergoing heart surgery, angioplasty, coronary bypass, and the like.

In other aspects of the invention, the method is carried out for the purpose of removing (or reducing the number of) connective tissue progenitor cells and/or other adhesive cell types from a sample, such as but not limited to a bone marrow sample. This is useful to isolate and/or enrich the non-adhesive cells within a sample, or simply to achieve physical separation between these cell types. Thus, the invention further contemplates methods on which the solution (rather than the microcarriers) is harvested from the vessel and administered to a subject, and the microcarriers are discarded.

Connective tissue progenitor cells are present in tissues to which they give rise including but not limited to the bone marrow, adipose tissue, cartilage, skin, muscle, and the like. The invention provides a method for isolating connective tissue progenitor cells from samples from these tissues, for example. The sample is therefore typically a bodily sample (i.e., it is a sample of bodily fluid such as blood, platelet rich plasma, or platelet poor plasma, or of bodily tissue such as those recited above). A bone marrow sample may be a bone marrow aspirate taken from a subject during a bone marrow harvest procedure. The harvest procedure typically comprises inserting a needle into the marrow space within the posterior ileac crest, and removing a sample. A harvest of bone marrow may comprise multiple separate entries into the marrow cavity in order to remove a sufficient amount of bone marrow.

The progenitor cells may be derived from adipose tissue. As used herein, adipose tissue refers to any fat tissue derived from any adipose tissue site, including brown or white adipose tissue. One of the advantages of using adipose tissue is that it is abundant and can be extracted from liposuction aspirates or from excised fat pads. Collagenase digestion can be used to release stem cells from the adipose tissue. (Halvorsen, et al, Metabolism 2001, 50:407-413; Hauner, et al, J Clin Invest 1989, 84:1663-1670; Rodbell, et al, J Biol Chem 1966, 241:130-139). A further advantage of using adipose tissue is that stem cells represent a higher proportion of the nucleated cell population within this tissue than within bone marrow (Sen, et al. Journal of Cellular Biochemistry 2001, 81:312-319).

In other embodiments, the sample may be extracted from skin. In certain embodiments, the source of skin may be neonatal foreskin. However, it should be appreciated that a skin sample could be taken from any part of the body of a subject. For example, methods for processing skin samples are described in US Patent Application Publication No. 20050106723, Hatzfeld et al., the contents of which are incorporated herein by reference. In some embodiments the skin sample is taken from a living subject. In other embodiments the skin sample can be taken from a cadaver.

In other embodiments, the sample may be extracted from muscle. A muscle sample could include any type of muscle such as skeletal muscle, smooth muscle and cardiac muscle. For example, methods of extracting and processing human skeletal muscle samples are described in US Patent Application Publication No. 20070123576, Shin et al., the contents of which are incorporated herein by reference. In one embodiment, the source of muscle may be the abductor hallucis muscle. However, it should be appreciated that a muscle sample could be taken from any part of the body of a subject.

In still other embodiments, the sample may be extracted from cartilage tissue. For example, methods of extracting and processing cartilage tissue are described in US Patent Application Publication No. 20040077079, Storgaard et al., the contents of which are incorporated herein by reference.

The sample may be taken from a human subject or from a non-human subject. In some embodiments a sample may be taken from an animal subject including but not limited to a horse or a cow. In other embodiments a sample may be taken from a companion animal subject such as but not limited to a dog or a cat.

In some embodiments, the sample may be an autologous sample. As used herein, an autologous sample refers to a sample that is harvested from the same subject to be treated. The connective tissue progenitor cell preparation generated according to the invention, for example, may be an autograft. In other embodiments, the sample may be an allogenic sample. As used herein, an allogenic sample refers to a sample that is harvested from a subject different from the subject to be treated. The donor and recipient subjects however are of the same species. The connective tissue progenitor cell preparation, as an example, may be an allograft. In some instances, the allograft may be syngeneic with the subject being treated (i.e., if the donor and recipient are genetically identical, as in the case of twins, for example). In other embodiments the sample may be a xenogeneic sample. As used herein, a xenogeneic sample refers to a sample that is harvested from a subject of a different species than the subject to be treated. As an example, the sample harvested from a pig (particularly some forms of genetically engineered pigs) may be intended for use in a human. In still other embodiments, the sample may be harvested from a cadaver or from a brain dead subject. In various of these embodiments, it may be necessary to tissue type the sample (or other sample from the donor, such as blood) in order to ensure maximal MHC compatibility with the recipient of the transplanted cells.

The sample may be a freshly explanted sample. As used herein, a freshly explanted sample refers to a sample that has been explanted from a subject within 5 hours and more preferably within 2 hours of manipulation according to the invention. In some embodiments, the freshly explanted sample has not undergone any manipulation outside of the vessel (e.g., it has not been processed outside of the vessel to remove cellular and/or extracellular components, and the like). As an example, in some embodiments it is preferable that the sample not be cultured prior to or after contact with the microcarriers. In some embodiments, the sample is maintained at room temperature. In some embodiments, the sample has not been diluted with medium or other buffers. In some instances, however, it may be desirable or necessary to manipulate the sample prior to contact with the microcarriers of the invention.

Aspects of the invention relate to cell carriers. In certain embodiments the cell carrier is a microcarrier. As used herein, a microcarrier is a single moiety capable of binding one or more cells such as connective tissue progenitors cells. Microcarriers are not limited in size to microns and rather can be larger or smaller than the micron range. In some embodiments, the microcarriers have a diameter that is about equal to or greater than the cells to which they bind. The microcarriers may therefore have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. In other embodiments, the microcarriers may be 50-200 μm, 75-200 μm, 100-200 μM, or 75-150 μm. Connective tissue progenitor cells range in size from about 5-30 microns.

It is to be understood that the terms microcarriers, microparticles and microbeads are used interchangeably herein, and that these terms embrace grains, granules, powder and the like, as used herein.

The microcarriers are able to bind connective tissue progenitor cells at least in part by virtue the adhesive phenotype of these cells, particularly as compared to other cells contained in the sample. Thus, in some instances connective tissue progenitor cells are more likely to bind to the microcarriers of the invention than are hemopoietic cells of the bone marrow, for example. In these instances, the progenitor cells will be preferentially retained on the microcarriers (and thereby harvested) as compared to the hematopoietic cells, in the case of a bone marrow starting sample. This will lead to an increased ratio of connective tissue progenitor cells to hematopoietic cells as compared to a starting bone marrow sample. This will be the case even if hematopoietic cells bind to the microcarriers, and even if they represent the majority of the cells bound to the microcarriers. It is to be understood therefore that, in some instances, the microcarriers harvested after the contacting step have no hematopoietic cells bound to them. In other instances, hematopoietic cells will be bound to the harvested microcarriers and these cells will represent less than about 80%, less than about 70%, less than about 60%, less than about 50%, less than about 40%, less than about 30%, less than about 25%, less than about 20%, less than about 15%, less than about 10%, or less than about 5% of the cells bound to the microcarriers.

In still another embodiment, it may be desirable prior to administration of the microcarriers to combine them with hematopoietic cells such as those present in the vessel solution, or a more defined population of hematopoietic cells in order to facilitate transplant and functioning of the progenitor cells.

It should be understood however that the microcarriers can bind other adhesive cells including adhesive differentiated cells of the bone, cartilage, skin, muscle or fat lineages.

Accordingly, the microcarriers administered to subjects may comprise connective tissue progenitor cells as well as differentiated cells present in the initial sample.

In most embodiments, the microcarriers are present as a plurality of microcarriers. As used herein, a plurality of microcarriers means two or more microcarriers. The plurality of microcarriers may be 2-10⁷ microcarriers, 2-10⁶ microcarriers, 2-10⁵ microcarriers, 2-10⁴ microcarriers, 2-10³ microcarriers, 2-10² microcarriers, or 2-10 microcarriers. The plurality may be 10²-10⁶ microcarriers, 10²-10⁵ microcarriers, 10²-10⁴ microcarriers, or 10²-10³ microcarriers.

It should be appreciated that microcarriers can be comprised of many different substances including but not limited to fibrin (as described in greater detail herein), chitosan, hyaluronic acid, collagen, PLGA, alginate, agarose, poly(ethylene glycol), dextran, methylcellulose, poly(acrylate), poly(ester), and the like. In some embodiments microcarriers may be comprised of extracellular matrix, such as autologous extracellular matrix. In these and other embodiments the microcarriers may be comprised of calcium phosphate, calcium carbonate, calcium sulphate, tricalcium phosphate, hydroxyapatite, and/or allograft bone such as allograft bone granules. It is to be understood that the microcarriers may comprise one or more of the foregoing substances and that therefore the microcarriers may comprise both organic and inorganic substances, although in some important embodiments the microcarriers are free of organic solvents and oil.

In some embodiments, the microcarriers have a core comprised of one or more substances and a coating comprised of one or more different substances. For example, the microcarriers comprise an organic core and an inorganic coat, such as but not limited to calcium phosphate.

It is to be understood that in some embodiments the invention contemplates the use of microcarriers in a suspension. As such, the majority of the microcarriers is not in physical contact with more than two microcarriers at the time of contact with the sample. In some embodiments, the majority of microcarriers is not in contact with more than one other microcarrier. In some embodiments, the majority of microcarriers is not in contact with any other microcarrier. As used herein, a majority of microcarriers means greater than 50%, including at least 55%, at least 60%, at least 65%, at least 70%, at least 75%, at least 80%, at least 85%, at least 90%, or at least 95% of microcarriers. “Microcarriers in solution”, as used herein, therefore are not compacted and do not form a matrix such as a unitary solid matrix. The microcarriers of the invention will be in flow during the contacting step of the isolation method rather than being fixed or static.

In some embodiments, the microcarriers are porous. A porous microcarrier can be a microcarrier having one or more channels that extend from its outer surface into the core of the microchannel. In some embodiments, the channel may extend through the microcarrier such that its ends are both located at the surface of the microcarrier. These channels are typically formed during synthesis of the microcarrier by inclusion followed by removal of a channel forming reagent in the microcarrier. These types of microcarriers may be referred to as non-hydrogel microcarriers.

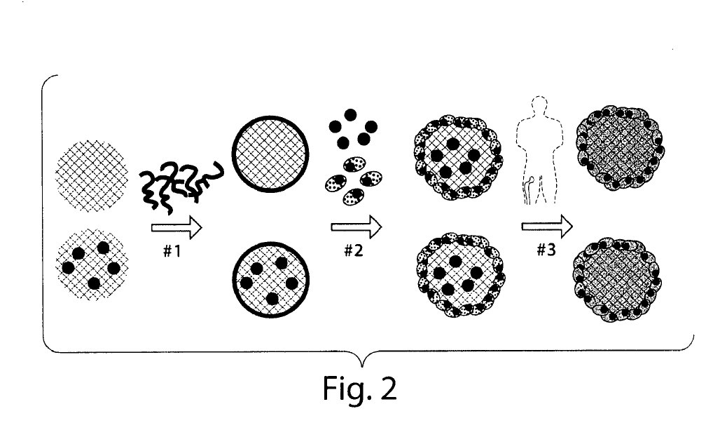

The size of the pores (or channels) may depend upon the size of the microcarrier. In certain embodiments, the pores (or channels) have a diameter of less than 15 microns, less than 10 microns, less than 7.5 microns, less than 5 microns, less than 2.5 microns, less than 1 micron, less than 0.5 microns, or less than 0.1 microns. In some instances, the pore (or channel) diameter is smaller than the cell diameter such as the connective tissue progenitor cell diameter. In these instances, the cell such as the connective tissue progenitor cell does not enter the pore (or channel) and is not located inside the carrier, and rather it is located on the carrier surface. Thus, it is to be understood that, in some instances, cells such as connective tissue progenitor cells are not located within the porous microcarriers. In other instances, however, the cells may enter the pores or channels partially or completely, and in doing so may be protected from shear from the dynamic (i.e., flow) environment of the contacting step of the isolation methods provided herein. (FIG. 2.)

The degree of porosity in porous microcarriers may range from greater than 0 to less than 100% of the microcarrier volume. The degree of porosity may be less than 1%, less than 5%, less than 10%, less than 15%, less than 20%, less than 25%, less than 30%, less than 35%, less than 40%, less than 45%, or less than 50%. The degree of porosity can be determined in a number of ways. For example, the degree of porosity can be determined based on the synthesis protocol of the carriers (e.g., based on the volume of the aqueous solution or other channel-forming reagent) or by microscopic inspection of the carriers post-synthesis.

A porous microcarrier can also be a microcarrier formed of a hydrogel. A hydrogel is a gel (or polymer) that is dispersed in an aqueous solution (e.g., water). The aqueous solution acts as a physical channel such that water-soluble agents external to the microcarrier can enter the microcarrier via partial or complete dissolution in the aqueous solution. The hydrogel may be comprised of collagen, fibrin, and/or hyaluronic acid, but it is not so limited. The hydrogel may be at least 5%, at least 10%, at least 15%, at least 20%, at least 25%, at least 30%, at least 35%, at least 40%, at least 45%, at least 50%, or more aqueous solution.

In some instances, the microcarriers are lyophilized (i.e., freeze-dried). Lyophilized microcarriers may be reconstituted with an aqueous solution that comprises one or more agents. The resulting reconstituted microcarriers will therefore comprise one or more agents.

The microcarriers may be solid (apart from channels or pores) or they may be hollow spheres. In some instances, the microcarriers comprise a degradable space filling cell invasive core (such as collagen). For these microcarriers, cells on the surface can migrate inward to promote 3-D cell-cell interactions and remodeling of the core into functional matrix (Overstreet et al., 2003, In Vitro Cell Dev Biol Anim 39, 228-234). Upon administration into a subject, the cells may integrate into the surrounding tissue and provide a bridge between new and existing tissue.

The plurality of microcarriers may be homogeneous for one or more parameters or characteristics. A plurality that is homogeneous for a given parameter, in some instances, means that microcarriers within the plurality deviate from each other no more than about +/−10%, preferably no more than about +/−5%, and most preferably no more than about +/−1% of a given quantitative measure of the parameter. As an example, the microcarriers may be homogeneously porous. This means that the degree of porosity within the microcarriers of the plurality differs by not more than +/−10% of the average porosity. In other instances, a plurality that is homogeneous means that all the microcarriers in the plurality were treated or processed in the same manner, including for example exposure to the same agent regardless of whether every microcarrier ultimately comprises the same agent. In still other embodiments, a plurality that is homogeneous means that at least 80%, preferably at least 90%, and more preferably at least 95% of microcarriers are identical for a given parameter (e.g., containing the same agent).

The plurality of microcarriers may be heterogeneous for one or more parameters or characteristics. A plurality that is heterogeneous for a given parameter, in some instances, means that microcarriers within the plurality deviate from the average by more than about +/−10%, including more than about +/−20%. Heterogeneous microcarriers may differ with respect to a number of parameters including their size or diameter, their shape, their composition, their surface charge, their degradation profile, whether and what type of agent is comprised by the microcarrier, the location of such agent (e.g., on the surface or internally), the number of agents comprised by the microcarrier, etc. The invention contemplates separate synthesis of various types of microcarriers which are then combined in any one of a number of pre-determined ratios prior to contact with the sample.

As an example, in one embodiment, the microcarriers may be homogeneous with respect to shape (e.g., at least 95% are spherical in shape) but may be heterogeneous with respect to size, degradation profile and/or agent comprised therein.

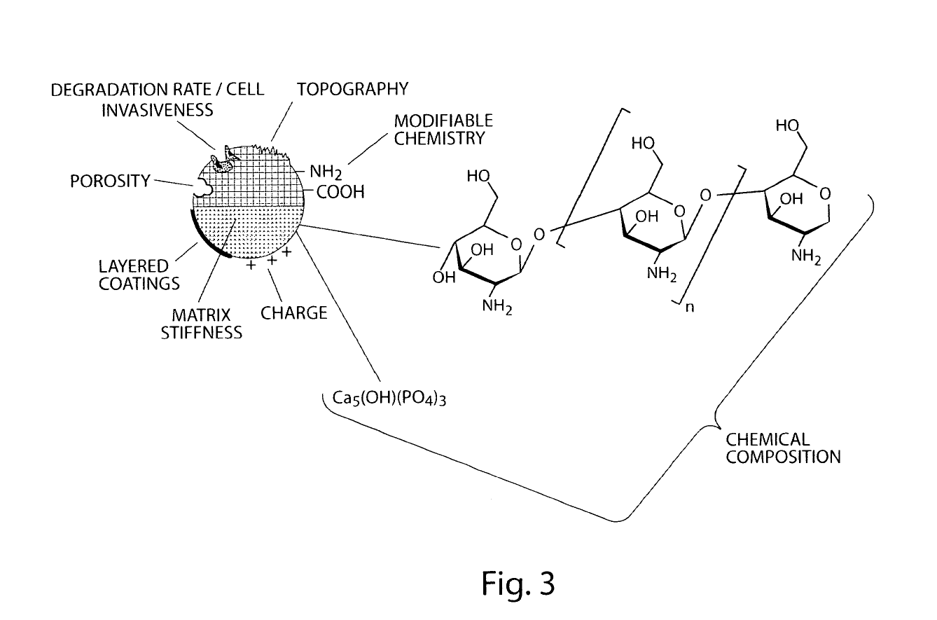

It should be appreciated therefore that many characteristics of the microcarriers can be modified to influence cell attachment and function in vivo post-administration. These characteristics include but are not limited to surface topography, surface chemistry, bulk chemical composition, charge density, matrix stiffness, layered coatings, porosity, and degradation rates. (FIG. 3.) In some embodiments, mechanical properties of the microcarrier can be modified to alter cell differentiation.

In other aspects, the invention provides compositions comprising fibrin microcarriers, methods of making such microcarriers, and methods of use thereof. The methods used for making these fibrin microcarriers are not dependent on oil-in-water emulsions and therefore they do not require the additional steps of oil removal which often times requires the use of organic solvents. Rather the invention provides in this aspect a method for making fibrin microcarriers that is simpler than the prior art methods presented by for example Gorodetsky and co-workers. One advantage of the fibrin microcarriers produced according to the methods of the invention is that such microcarriers are free of organic solvents and/or oil and therefore they are would be considered a priori more compatible and suitable for human use. The fibrin microcarriers described herein can be used for any number of applications, including but not limited to the bioreactor attachment and enrichment methods provided herein.

Thus, the invention provides a method for making a fibrin microcarrier composition by combining (or mixing or physically contacting) fibrinogen and thrombin to form fibrin, as a first step. Fibrinogen and thrombin are typically both provided as solutions, and the concentrations of these solutions may vary. The Examples provide a synthesis scheme in which equal volumes of fibrinogen solution and thrombin solution are combined to give a final concentration of 5.4 mg/ml fibrinogen and 2.5 U/ml thrombin. Other final concentrations are also contemplated ranging from 0.1-40 mg/ml fibrinogen. Fibrinogen and thrombin may be purchased commercially from suppliers such as Sigma (fibrinogen is F8630-1G, and thrombin is T6634-500UN). In the embodiment illustrated in the Examples, the fibrinogen solution is added drop wise to the thrombin solution through a needle while the thrombin solution is agitated (e.g., using sonication). The fibrinogen and thrombin solutions are generally aqueous in nature, thereby giving rise to the resultant fibrin hydrogel.

This hydrogel is then dehydrated using any number of methods. This step may be referred to as dehydrating, drying or desiccating the hydrogel, all of which refer herein to the process of removing as much of the water content from the hydrogel as possible. Dehydration can therefore be accomplished using heat, vacuum, lyophilization, desiccation, and the like. In some embodiments, lyophilization may be preferred since microcarriers that result therefrom are less likely to swell once in contact with an aqueous solution. The dehydration step may occur over a range of time, depending on the particular method used and the volume of the hydrogel. For example, the step may last for a few minutes, a few hours, or a few days. The invention is not intended to be limited in this regard.

Once the hydrogel is dehydrated, it is ground to form microcarriers. The dehydrated hydrogel may be ground, pulverized, milled, crushed, granulated, pounded, and the like, provided the effect is to produce microcarriers. As shown in the Examples, the microcarriers may be prepared using a mortar and pestle.

Importantly, the method does not require emulsions and thus does not involve removal of oils with for example organic solvents. The method may therefore be referred to herein as a “non-emulsion” method. The fibrin microcarriers produced are therefore free of oil and also free of organic solvents. The presence of oil and/or organic solvents may be determined through a chemical analysis of the microcarriers. As an example, the microcarriers may be placed in an aqueous or organic solution, thereby allowing their constituents to leach out and into the solution. The solution can then be tested for the presence of oil and/or organic solvents using for example mass spectrometry, HPLC, or any other suitable chemical analysis method, as will be known to those of ordinary skill in the art.

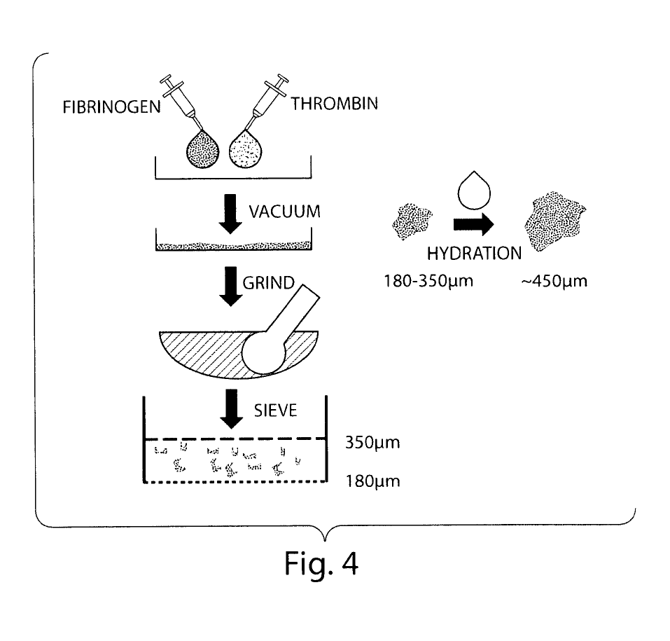

The method may further involve size separating the fibrin microcarriers. This may be accomplished most easily by sieving the microparticle composition through one or more appropriate sieves or filters having desired pore sizes. FIG. 4 demonstrates for example sieving of microcarriers through a 350 μm pore sieve, followed by a 180 μm pore sieve. In this way, at least three populations of microcarriers can be isolated from the bulk population and these are a first population that is retained by the 350 μm pore sieve, a second population that is retained by the 180 μm pore sieve, and a third population that is retained by neither sieve. The first population is generally regarded herein as a population having an average diameter of greater than 350 μm, the second population is generally regarded herein as a population having an average diameter between 180-350 μm, and the third population is generally regarded herein as a population having an average diameter of less than 180 μm. These populations and size ranges are meant to be illustrative only. In some embodiments the microcarriers are further sieved to arrive at populations having average diameters in the range of 85-180 μm. In still other embodiments, the microcarriers are 90-170, 100-160, 100-150, 110-150, 120-140, or about 130 μm in average diameter. The microcarriers may be equal to or less than 80, 90, 100, 110, 120, 130, 140, 150, 160, 170, or 180 μm, provided they have a minimum average diameter of at least 10, 20, 30, 40 or 50 μm. It is to be understood that these average diameters refer to the diameter of the dehydrated microcarriers rather than their rehydrated diameters. The microcarrier volume may increase 10-250% of the initial volume after rehydration.

In some embodiments, fibrin microcarriers are provided that are size restricted. In some aspects, the composition comprises a plurality of fibrin microcarriers, wherein at least 50% of which have an average diameter of 85-180 μm prior to hydration. In some embodiments, at least 55%, at least 60%, at least 65%, at least 70%, at least 75%, at least 80%, at least 85%, at least 90%, at least 95%, or more of the fibrin microcarriers have an average diameter of 85-180 μm.

The microcarriers produced by this method are typically of irregular shape and size. That is, for the most part these microcarriers are not spherical. As a result, the diameter of each microcarrier is determined by summing its longest and its shortest dimension and dividing that sum by two. This is referred to as the average diameter of a single microcarrier. Average diameter of a population of microcarriers may be deduced based on a sieving analysis such as that described above (i.e., the sieving analysis would provide a range of average diameters based on retention and/or flow through of microcarriers).

In some embodiments, the fibrin microcarriers are also exposed to a cross-linking agent in order to cross-link fibrin strands and thereby increase the half-life of the microcarriers in vivo. While the invention contemplates the use of any cross-linking agent, those that are physiologically compatible are preferred in most embodiments. An example of such a cross-linking agent is Factor XIII. It has been found according to the invention that fibrin microcarriers that are crosslinked using Factor XIII have a half-life of about 13 weeks in vivo while those that are not crosslinked have a half-life of about 7 days. Fibrin cross-linking can be assessed by the number of alpha and gamma fibrin monomers, wherein the number decreases upon cross-linking. It can also be assessed by the presence and number of gamma-gamma crosslinks (for example as can be observed using reducing SDS PAGE.

In some embodiments, fibrin microcarriers are provided that are defined by their surface topology, topography, or roughness. This surface topology or roughness may be expressed in terms of the number and/or size of features (or protrusions) on the surface of microcarriers. Roughness can be observed using techniques commonly used in the art including optical profilimetry and atomic force microscopy. The number of features on these microcarriers may range from 2-100 typically. The size of these features (or protrusions) may be expressed in terms of absolute length or in terms of the ratio of the size of the feature (or protrusion) and the average diameter of the microcarrier. In some embodiments, the size of the feature is about 1 μm, about 2 μm, about 3 μm, about 4 μm, about 5 μm, about 6 μm, about 7 μm, about 8 μm, about 9 μm, about 10 μm, or more. In other embodiments, the size of the feature is more than 10 μm, more than 15 μm, more than 20 μm, more than 25 μm, more than 30 μm, more than 35 μm, more than 40 μm, more than 45 μm, more than 50 μm, or more. In still other embodiments, the size is 10-100 μm. In other embodiments, the size is 1-10 μm. The ratio of feature size and microcarrier average diameter may be about 5%, 10%, 15%, 20%, 25%, 30%, 35%, 40%, 45%, or 50%, or more. This surface roughness is important since it has been found that cells such as connective tissue progenitor cells are better able to bind to microcarriers having a greater degree of surface roughness.

In some embodiments, the fibrin microcarriers are coated with a cell adhesion molecule that promotes attachment of cells to the microcarriers. Since most cells are inherently negatively charged at their surface, cell adhesion molecules that are positively charged can be used to attach most cells to the microcarriers. An example of a positively charged cell attachment agent is poly-lysine, such as poly-D-lysine or poly-L-lysine. As described in the Examples, adding poly-lysine to the thrombin solution prior to (or during or after) adding the fibrinogen solution generates a fibrin hydrogel that comprises poly-lysine. The concentration of poly-lysine in the final solution may range from 100-1000 μg/mL. The fibrin-lysine hydrogel can be dehydrated and then ground as described herein. Once the microcarriers are made, they may be rinsed in an aqueous solution (such as but not limited to phosphate buffered saline, PBS) to remove excess poly-lysine. The resultant fibrin microcarriers comprise poly-lysine, and are not typically not limited to being simply surface coated with poly-lysine.

The compositions of microcarriers may further comprise a pharmaceutically acceptable carrier, one or more agents such as therapeutic agents, and in some instance may further comprise cells. The nature of the cells will depend on the intended use of the microcarriers complexed with cells. In some embodiments, the cells may be mammalian cells, preferably but not limited to human cells. These cells may be connective tissue progenitor cells, hematopoietic progenitors, endothelial progenitor cells, or other progenitor or end stage cells.

The invention therefore contemplates methods for attaching cells to the fibrin microcarriers. The fibrin microcarriers are contacted with the cells of interest, or with a population of cells that contains the cells of interest, for a time and under conditions sufficient for the cells to bind to the fibrin microcarrier. If the microcarriers comprise lysine (or poly-lysine), then typically cells will attach rapidly and non-specifically. Attachment in this instance can occur within a hour, within 30 minutes, or within 10 minutes. The cells and microcarriers may be combined in a physiological and/or a pharmacological carrier such as but not limited to sterile PBS or a cell medium. Preferably, this incubation occurs in a controlled environment with respect to temperature, CO₂ level, pH, and the like.

In some embodiments, the cells and microcarriers are contacted in vitro, such as in sterile container or vessel such as a tissue culture vessel. The contacting therefore may occur as part of an in vitro culture of the cells or tissue from which the cells derived. In other embodiments, the contacting may occur in vivo by introducing the microcarriers into a subject and allowing cells to attach to the microcarriers. The microcarriers may be introduced into the subject alone or in the context of a delivery vehicle such as an implant that comprises the microcarriers.

In certain embodiments, the microcarriers are used to aggregate cells.

The microcarriers may comprise one or more agents. The agent(s) may be located (e.g., incorporated) within the microcarrier (e.g., within pores or channels of the microcarrier) and/or on the external surface of the microcarrier. In some instances, the microcarriers are pre-loaded with the one or more agents (i.e., they already comprise the one or more agents prior to contact with the samples). The agents include but are not limited to growth factors, cell adhesion molecules, anti-microbial agents, anti-inflammatory agents, immunosuppressive agents, and inhibitors of bone resorption. Inhibitors of bone resorption include but are not limited to proton pump inhibitors, bisphosphonates, e.g. clodronic acid, etidronic acid, pamidronic acid, aledronic acid, ibandronic acid, zoledronic acid, risedronic acid or tiludronic acid, and salts and hydrates thereof, steroid hormones e.g. estrogen, a partial estrogen agonist or estrogen-gestagen combination, a SERM (Selective Estrogen Receptor Modulator) e.g. raloxifene, lasofoxifene, TSE-424, FC1271, Tibolone (Livial®), tamoxifene, droloxifene, toremifene, idoxifene, or levormeloxifene), calcium, a calcitonin-like substance or derivative thereof, e.g. salmon, eel or human calcitonin, vitamin D or an analog thereof, alendronate sodium, etidronate disodium, pamidronate disodium, or an activator of parathyroid hormone (PTH) release.

As used herein, the term growth factor refers to any agent that stimulates cellular proliferation and/or differentiation. Growth factors of particular interest for the methods of the invention include but are not limited to those that stimulate production of bone by stimulating osteogenic differentiation, collagen production, mineralization, and osteogenic cell recruitment, and/or by suppressing osteoclast number and/or function, those that stimulate hematopoietic development and differentiation, or those that stimulate endothelial development and differentiation. Growth factors include but are not limited to fibroblast growth factor (FGF), platelet-derived growth factor (PDGF), insulin-like growth factors (IGF) I and II, TGF-β, TGF-α, bone morphogenetic protein (BMP) (e.g., BMP-2, BMP-3, BMP-4, BMP-6, or BMP-7), hedgehog proteins, growth differentiation factors, hematopoietic colony-stimulating factors (CSF), vascular endothelium growth factor (VEGF), osteoid-inducing factor (OIF), angiogenins, endothelins, hepatocyte growth factor, keratinocyte growth factor, ADMP-1, interleukins (IL) (e.g., IL-3 and IL-6), epithelial growth factors, dexamethasone, leptin, sortilin, transglutaminase, prostaglandin E, 1,25-dihydroxyvitamin D3, ascorbic acid, pro-collagen, glycerol phosphate, TAK-778, statins, growth hormone, steel factor (SF), activin A (ACT), retinoic acid (RA), epidermal growth factor (EGF), hematopoietic growth factors, peptide growth factors, erythropoietin, tumor necrosis factors (TNF), interferons (IFN), heparin binding growth factor (HBGF), nerve growth factor (NGF) and muscle morphogenic factor (MMP).

For example, TGF family members stimulate production of extracellular matrix characteristic of cartilage tissue. Bone morphogenetic protein, colony stimulating factors specific to bone, and/or PDGF stimulate production of collagen and other bone ECM proteins. Dexamethasone may stimulate osteogenic differentiation, and ascorbic acid may promote development of osteoclasts. Adipocyte formation may be stimulated with dexamethasone and insulin, and skeletal muscle cell differentiation may be stimulated with 5-azacytidine. High concentrations of PDGF in serum-free media without other growth factors can stimulate development of smooth muscle cells, and substitution of b-FGF for PDGF can stimulate cardiac muscle cell formation.

It will be understood that the amount of growth factor required according to the invention is lower, and in most cases far lower, than that which would be required if administered independently of the microcarrier. In some instances, the amount will be at least 2-fold, at least 5-fold, or at least 10-fold lower than the microcarrier-independent administration amount. It will also be understood that the invention also contemplates the administration of agents such as therapeutic agents that are not comprised in microcarriers, although they may be formulated together with or administered together with the microcarriers of the invention.

In some embodiments, the microcarriers are designed to comprise cell adhesion molecules, preferably on their external surface. These molecules enhance the binding of cells such as progenitor cells to the microcarriers. As used herein, a cell adhesion molecule is any molecule that can be attached to (including coated onto or incorporated within) the microcarriers of the invention and to which cells such as connective tissue progenitor cells can bind. The cell adhesion molecule may be capable of binding both cells and extracellular matrix in some embodiments. These molecules may be organic in nature, in which case they may be peptides (or proteins), carbohydrates and/or polysaccharides, lipids, nucleic acids such as aptamers, and the like. In other embodiments, they may be non-organic in nature in which case they may be, for example, chemical compounds. In still other embodiments, they may be a combination of one or more organic molecules and/or one or more non-organic molecules. Examples of cell adhesion molecules include but are not limited to peptides or proteins such as RGD-containing peptides, antibodies or antibody fragments, and cell adhesion proteins such as but not limited to fibronectin, collagen, laminin, gelatin (denatured collagen), vitronectin, vascular cell adhesion molecule (V-CAM), intercellular adhesion molecule (I-CAM), tenascin, thrombospondin, osteonectin, osteopontin, bone sialoprotein, cadherins, integrins, lectin, neural cell adhesion proteins, some members of the proline-rich proteins, poly-lysine, hyaluronic acid, and ceramics. In some embodiments, microcarriers are coated with cell adhesion molecules, for example, post-synthesis.

Some cell adhesion molecules may function as ligands for particular cells in the sample. Other cell adhesion molecules may function as ligands for particular cells in vivo. In either case, the ligands allow the adhesion of desired cell types to the microcarriers.

Anti-microbial agents are agents that inhibit the growth of or kill microbes such as bacteria, mycobacteria, viruses, fungi, and parasites. Anti-microbial agents therefore include anti-bacterial agents, anti-mycobacterial agents, anti-viral agents, anti-fungal agents, and anti-parasite agents. Examples of these agents can be found in US Patent Application Publication No. 20050084490. Microcarriers so loaded can be used in joint replacement therapies to prevent or control infection.

Anti-inflammatory agents are agents that reduce or eliminate inflammation. Examples include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lornoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, and zomepirac sodium.

Immunosuppressive agents are agents that reduce or eliminate an immune response. Immunosuppressive agents are typically used in a transplant setting in order to prevent graft versus host disease or host versus graft disease. Examples include azathioprine, azathioprine sodium, cyclosporine, daltroban, gusperimus trihydrochloride, sirolimus, rapamycin, mycophenolic acid, mycophenolate mofetil, riboflavin, tiazofurin, methylprednisolone, zafurin, methotrexate, antisense and antibodies against ICAM-1, glucocorticoids, anti-CD25 basiliximab, daclizumab, tacrolimus (FK-506), pimecrolimus, mTOR inhibitors, mizoribine, interferons, corticosteroids, cyclophophamide, sulfasalazine, anti-eukocyte antibodies (such as CAMPATH-1), and the like.

In certain embodiments, the release of agents impregnated within the microcarriers may be controlled through modifying the concentration of matrix within the microcarrier or the porosity of the microcarrier. In some embodiments, microcarriers may be combined with micro- or nano-particle based drug delivery platforms to achieve a variety of release profiles. In certain embodiments, the microcarriers may be pre-encapsulated within a hydrogel material such as collagen, dextran, alginate, PEG or hyaluronic acid, in order to reduce the interaction of cells such as connective tissue progenitor cell with the host environment after transplant.

The isolated cells such as the isolated connective tissue progenitor cells (and/or microcarriers) may be formulated as pharmaceutical compositions or preparations for use in vivo. A pharmaceutical preparation is a composition suitable for administration to a subject. Such preparations are usually sterile and prepared according to GMP standards, particularly if they are to be used in human subjects. In general, a pharmaceutical composition or preparation comprises the microcarriers, cells, and optionally agent(s) of the invention and a pharmaceutically-acceptable carrier, wherein the cells and agent(s) are generally provided in effective amounts. The compositions may comprise agents by virtue of the fact that the microcarriers may contain agents. Alternatively or additionally the preparation may also comprise one or more agents independent of the microcarriers. These agents include but are not limited to one or more growth factors, anti-microbial agents, anti-inflammatory agents, immunosuppressive agents, and inhibitors of bone resorption. In preferred embodiments, the compositions are formulated for injection. These pharmaceutical preparations are in some instances formulated for delivery using a needle and syringe, preferably where the needle is a 14 gauge or smaller needle.

As used herein, a pharmaceutically-acceptable carrier means a non-toxic material that does not interfere with the effectiveness of the biological activity of the compositions of the invention. Pharmaceutically-acceptable carriers include diluents, fillers, salts, buffers, stabilizers, solubilizers and other materials which are well-known in the art. Such preparations may routinely contain salt, buffering agents, preservatives, compatible carriers, and optionally other therapeutic or prophylactic agents. When used in medicine, the salts should be pharmaceutically acceptable, but non-pharmaceutically-acceptable salts may conveniently be used to prepare pharmaceutically-acceptable salts thereof and are not excluded from the scope of the invention. Such pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compositions of the invention may be formulated into preparations in solid, semi-solid, liquid or gaseous forms, and usual ways for parenteral or surgical (e.g., by washing or bathing of the surgical site) administration. The invention also embraces pharmaceutical compositions which are formulated for local administration, such as by local injection or implants.

The compositions may be administered directly to a tissue. Preferably, the tissue is one that is likely to respond beneficially to the compositions of the invention. Direct tissue administration may be achieved by direct injection including but not limited to arthroscopic injection. In some embodiments, the compositions are administered by injection using 14, 16, 18, 20, 21, 23, 25 or smaller gauge needles.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, and inert gases and the like.

Other delivery systems can include time-release, delayed release or sustained release delivery systems. Many types of release delivery systems are available and known to those of ordinary skill in the art. They include polymer based systems and non-polymer based systems such as lipids including sterols such as cholesterol, cholesterol esters and fatty acids or neutral fats such as mono-, di-, and tri-glycerides; hydrogel release systems, silastic systems, peptide based systems, wax coatings, compressed, tablets using conventional binders and excipients, partially fused implants; and the like. Specific examples include but are not limited to (a) erosional systems in which agents such as growth factors are contained within a matrix such as those described in U.S. Pat. Nos. 4,452,775, 4,675,189, and 5,736,152, and (b) diffusional systems in which agents such as growth factors permeate at a controlled rate from a polymer such as described in U.S. Pat. Nos. 3,854,480, 5,133,974 and 5,407,686. In addition, pump-based hardware delivery systems can be used, some of which are adapted for implantation.

In some embodiments, the delivery vehicle is a gel implant comprising the microcarrier. Exemplary bioerodible implants that are useful in accordance with this method are described in PCT International Application No. PCT/US/03307 (Publication No. WO 95/24929, entitled “Polymeric Gene Delivery System,” claiming priority to U.S. Pat. No. 213,668, filed Mar. 15, 1994). PCT/US/0307 describes a biocompatible, preferably biodegradable polymeric matrix for containing a biological macromolecule. Forms of the polymeric matrix for containing the microcarriers include films, coatings, gels, implants, and stents. The size and composition of the polymeric matrix is selected to result in physical compatibility and in favorable release kinetics in the tissue into which the matrix is implanted. The size of the polymeric matrix may be further selected according to the method of delivery which is to be used.

Both non-biodegradable and biodegradable polymeric matrices can be used to deliver the microcarriers, cells and agents to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the microcarriers, cells and/or agents of the invention may be delivered using the bioerodible implant by way of diffusion, or more preferably, by degradation of the polymeric matrix.

A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference). Other exemplary polymers for forming either two dimensional or three dimensional scaffolds include PLA, PGA, PLGA, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes, polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of any of the above, adducts of any of the above, and mixtures of any of the above polymers, co-polymers, and adducts with one another.

Exemplary synthetic polymers which can be used to form the biodegradable delivery system include polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polymers of lactic acid and glycolic acid, polyanhydrides, poly(ortho)esters, polyurethanes, poly(butic acid), poly(valeric acid), and poly(lactide-cocaprolactone) and polyvinylpyrrolidone.

Non-polymer materials, for example, may also be employed as both two and three-dimensional substrates. Exemplary materials include alumina, calcium carbonate, calcium sulfate, calcium phosphosilicate, sodium phosphate, calcium aluminate, calcium phosphate, hydroxyapatite, tricalcium phosphate, dicalcium phosphate, tricalcium phosphate, tetracalcium phosphate, amorphous calcium phosphate, octacalcium phosphate, and silicates.

Examples of biodegradable polymers include natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Examples of non-biodegradable polymers include ethylene vinyl acetate, poly(meth)acrylic acid, polyamides, copolymers and mixtures thereof.

Polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Biologically-compatible polymers includes but are not limited to propylene glycol, polypropylene glycol, polyethylene glycol acrylic acid cross-linked with polyallyl sucrose, an organic polymer acid colloid, a polyuronic acid, a carboxypolymethylene compound, a polyester resin containing three carboxyl group's, a partially hydrolyzed polyacrylate, polymethacrylates, polyoxyethylenes, polypropylene copolymers, polysaccharides such as cellulose, ethylcellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, starch, flour and mixtures thereof.

In still another aspect, the invention contemplates the use of the microcarriers described herein in various prior art devices and methods including those described in U.S. Pat. Nos. 5,824,084 and 6,049,026. In this aspect, the instant microcarriers would replace the bone graft substitute contemplated by the prior art. One advantage of this substitution is that the microcarriers can modulate the functioning of the progenitor cells once transplanted more so than the inert bone graft substitutes of the prior art.

The invention further contemplates kits comprising elements of the invention. In one aspect, the kit comprises a sterile vessel comprising an inlet/outlet port (or separate inlet and outlet ports) and a stirrer, and instructions for isolating connective tissue progenitor cells from a sample according to the methods provided herein.

In another aspect, the kit comprises a sterile vessel comprising an inlet/outlet port and a stirrer, a first container comprising a plurality of sterile microcarriers, a second container comprising one and optionally more agents, and a third container comprising a sterile solution. The kit optionally contains instructions for isolating connective tissue progenitor cells from a sample according to the methods provided herein. The kit may further comprise additional containers each of which may comprise one or more additional agents. The kit may also comprise a filter system that can be used to harvest the microcarriers post-contacting with sample. The vessel may be disposable.

### EXAMPLES

### Example 1

### Fibrin Microcarrier Synthesis

Microcarriers have been made from crosslinked dextran (Cytodex). This material however is not degradable and therefore not suitable for in vivo applications. Examples of biodegradable materials used in microcarriers are poly lactic-co-glycolic acid (PLGA), poly-(L)-lactic acid (PLLA) and hydroxyapatite. (Malda et al. In Vitro Cell Dev Biol. Anim. 2003 39(5-6):228-234.) PLGA and PLLA are degradable materials however cells cannot remodel these polymers easily and thus the volume available for tissue growth is minimal until the beads have sufficiently degraded by hydrolysis.

One of the challenges in creating hydrogel-based microcarriers is the requirement to use an oil-in-water emulsion for creation of the particles. Although biocompatible oils can be used, the oil needs to be removed in the final stages of microcarrier manufacture and this process requires use of organic solvents. For example, for creation of fibrin beads, a solution of fibrinogen and thrombin is added into a stirred preheated oil suspension. The aqueous protein solution will form oil-suspended droplets. Mixing and heating continues for approximately 6 hours which results in the evaporation of the aqueous phase and formation of fibrin particles. The cross-linked fibrin beads are then rinsed from oil residues in an organic solvent and dried. (Kassis et al. Bone Marrow Transplantation 2006, 37:967-976; Zangi et al. Tissue Engineering 2006, 12(8):2343-2354; Gorodetsky et al. J Invest Dermatol. 1999, 112(6); Gurevich et al. Tissue Engineering 2002, 8(4); Rivkin 2007, Cloning and Stem Cells 9(2); Shimony 2006, Kidney International, 69:625-633.

The invention has overcome these various challenges by providing methods for synthesizing fibrin microcarriers from dehydrated fibrin hydrogels. This method does not require oil or organic solvents in the manufacture of the microcarriers. It has been found according to the invention that cells rapidly attach to the microcarriers and grow on them, and therefore these microcarriers are suitable for various therapeutic applications.

As an exemplary material we have used fibrin to create fibrin microcarriers (FMC). The process involves addition of fibrinogen solution to a thrombin solution which quickly leads to a gel (FIG. 4). The hydrogel that forms is then dried in a desiccator, crushed and ground, with mortar and pestle. The FMC are then size separated by sieving. To enhance rapid attachment of cells, FMC can be treated with cell adhesive proteins or peptide sequences or with positively charged agents such as poly-lysine. When hydrated, the fibrin FMC will swell by 50-100% of their original volume.

### Example 2

### Microcarrier Testing

To demonstrate a proof of concept that these FMC can accommodate cells, FMC with or without poly lysine were placed into 15 mL bioreactors with mesenchymal stem cells. Human mesenchymal stem cells were purchased from Tulane Center for Gene Therapy (New Orleans, La.). Human adult stem cells were recovered from frozen vials containing approximately one million cells. Lentivirus transduced MSCs with cytoplasmic expression of green fluorescent protein (GFP) were also used in this study to facilitate rapid detection and visualized cell attachment to fibrin microbeads using fluorescent microscopy.

Untreated FMC required 2 hours for 50% cells to attach with addition of 1250 cells/mg FMC and incubating at 37° C. and 120 rpm. The total cell suspension volume in the bioreactor was 500 μL. The cell attachment was determined by taking a sample of the suspension after letting the FMC sediment for about 15 sec. The sample was then transferred to a well of a 96-well plate, media was added and after 20-24 hours the cell samples on the plates were fixed in formaldehyde. The number of cells in 5 random fields was counted using phase contrast microscopy and the concentration of unattached cells in the suspension was determined.

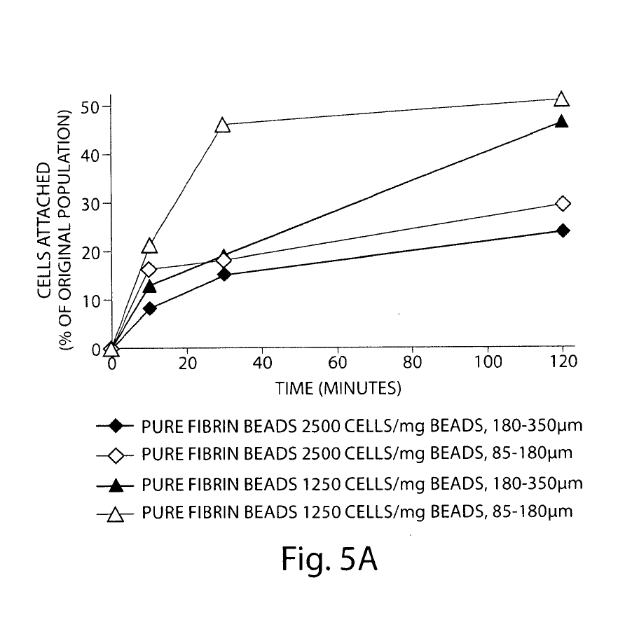

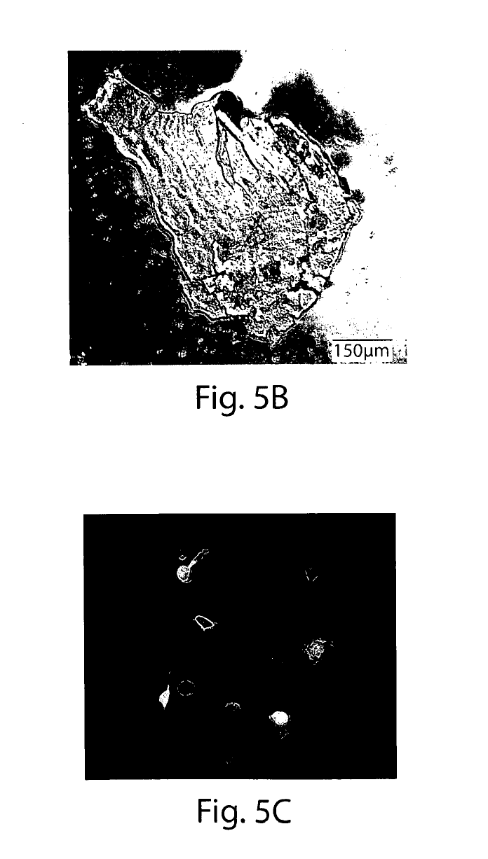

The cell attachment rate for different cell-to-FMC ratios is shown in FIG. 5A. Cell attachment is nearly doubled by using a higher FMC/cell ratio. The cell carriers created with this method are significantly different from other previous described. Instead of being spherical, they are very irregular in shape with sharp edges and corners as seen in FIG. 5B. FMC selected in the size range of 180-350 μm in their dry state swell to a size of 478±26 μm after hydration. Cells quickly adhere to the surface of the fibrin FMC and stretched out cells can be observed after just 120 minutes as seen in FIG. 5C.

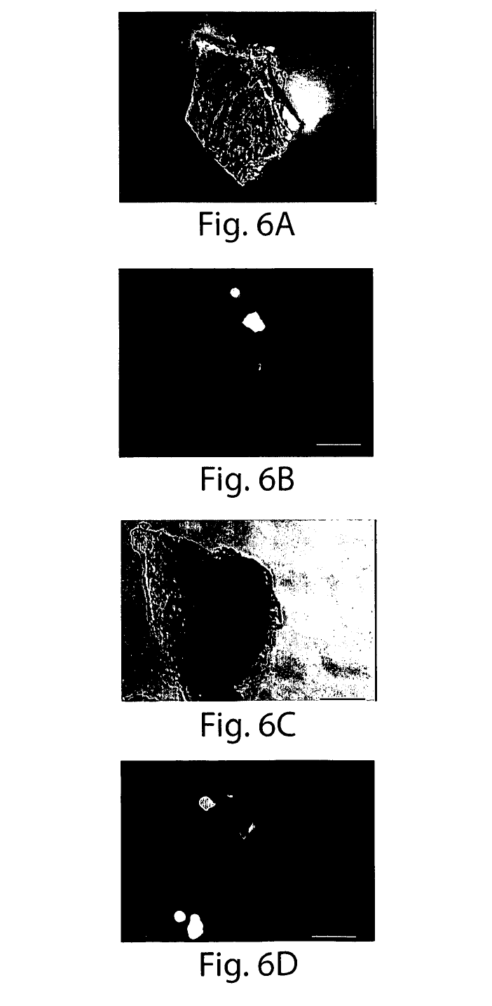

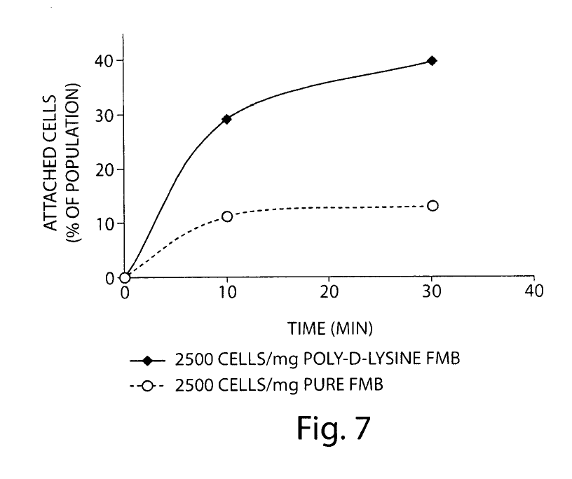

Poly-lysine, both poly-D-lysine (PDL) and poly-L-lysine (PLL), are examples of positively charged molecules that are commonly used to enhance cell attachment to plastic, glass surfaces and other materials. Poly-lysine surface treatment improves adhesive properties by adding a net positive charge and has been utilized to functionalize the surface of cell carriers for adherent cell populations like mesenchymal progenitor cells and fibroblasts. For example poly lactide co-glycolide microcarriers that incorporated poly-lysine were shown to facilitate enhanced cell attachment. (Chun et al. Biotechnol. Prog. 2004, 20(6):1797-801.) In the case of mesenchymal stem cells, cell attachment to the beads treated with poly-D-lysine increased by ˜300% within 30 min, compared to pure FMB, as shown in FIGS. 6 and 7 below.

### EQUIVALENTS

It should be understood that the preceding is merely a detailed description of certain embodiments. It therefore should be apparent to those of ordinary skill in the art that various modifications and equivalents can be made without departing from the spirit and scope of the invention, and with no more than routine experimentation.

All references, patents and patent applications that are recited in this application are incorporated by reference herein in their entirety.

What is claimed is:

1. A method for isolating cells from a sample comprising
contacting a sample with a plurality of microcarriers in solution under conditions and for a time sufficient for cells in the sample to bind to the microcarriers, and
harvesting from the solution microcarriers bound to cells,
wherein the sample is not cultured in vitro prior to or after contact with the microcarriers, and wherein the majority of microcarriers within the plurality is porous.

2. A method for isolating cells from a sample comprising
contacting a sample with a plurality of microcarriers in solution under conditions and for a time sufficient for cells in the sample to bind to the microcarriers, and
harvesting from the solution microcarriers bound to cells,
wherein the sample is not cultured prior to or after contact with the microcarriers, and the majority of microcarriers in solution is not in contact with more than one other microcarrier.

3. The method of claim 1, wherein the cells are connective tissue progenitor cells.

4. The method of claim 1, wherein the cells are hematopoietic progenitor cells.

5. The method of claim 1, wherein the cells are endothelial progenitor cells.

6. The method of claim 1, wherein the sample is contacted with the plurality of microcarriers in a vessel.

7. The method of claim 1, wherein the sample and the plurality of microcarriers in solution are continuously stirred during the contacting step.

8. The method of claim 1, wherein the microcarriers are harvested from the solution using density based separation.

9. The method of claim 1, wherein the microcarriers are harvested from the solution using filtration.

10. The method of claim 1, wherein the sample and solution are in a volume ratio of about 1:50.

11. The method of claim 1, wherein the sample is a freshly explanted sample.

12. The method of claim 1, wherein the sample is a tissue sample.

13.-53. (canceled)

54. A method for transplanting cells into a subject comprising
isolating cells from a sample according to the method of claim 1, and
administering the cells into a subject in need thereof.

55.-59. (canceled)

60. A kit comprising
a sterile vessel comprising an inlet/outlet port and a stirrer, and
instructions for isolating cells from a sample according to the method of claim 1, wherein the microcarriers are contacted with the sample in the vessel.

61. A kit comprising
a sterile vessel comprising an inlet/outlet port and a stirrer,
a first container comprising a plurality of sterile microcarriers,
a second container comprising one or more agents, and
a third container comprising a sterile solution, and optionally
instructions for isolating cells from a sample according to the method of claim 1.

62.-65. (canceled)

66. A pharmaceutical composition comprising
cells prepared according to the method of claim 1 bound to one or more microcarriers, and
a pharmaceutically acceptable carrier.

67.-74. (canceled)

75. A non-emulsion method for making a fibrin microcarrier composition, comprising
combining fibrinogen and thrombin to form a fibrin hydrogel,
dehydrating the fibrin hydrogel, and
grinding the dehydrated fibrin hydrogel to produce fibrin microcarriers.

76.-83. (canceled)

84. A composition comprising
a plurality of fibrin microcarriers, at least 50% of which have an average diameter of 85-180 μm prior to hydration.

85.-97. (canceled)

98. A method for producing cell-bound fibrin microcarriers comprising
contacting a fibrin microcarrier made according to the method of claim 75 with a cell for a time and under conditions sufficient for the cell to bind to the fibrin microcarrier.

99. A method for producing cell-bound fibrin microcarriers comprising
contacting a population of fibrin microcarriers of claim 84 with cells for a time and under conditions sufficient for the cells to bind to the fibrin microcarriers.

100.-108. (canceled)
